SEQUENCE LISTING (6 sequences)

SEQ ID NO: 1 - protein, 3 aa, Artificial Sequence
  Description of Artificial Sequence; Note = synthetic construct
RGD

SEQ ID NO: 2 - protein, 4 aa, Artificial Sequence
  Description of Artificial Sequence; Note = synthetic construct
DGEA

SEQ ID NO: 3 - protein, 5 aa, Artificial Sequence
  Description of Artificial Sequence; Note = synthetic construct
YIGSR

SEQ ID NO: 4 - protein, 4 aa, Artificial Sequence
  Description of Artificial Sequence; Note = synthetic construct
GPRP

SEQ ID NO: 5 - protein, 6 aa, Artificial Sequence
  Description of Artificial Sequence; Note = synthetic construct
KQAGDV

SEQ ID NO: 6 - protein, 5 aa, Artificial Sequence
  Description of Artificial Sequence; Note = synthetic construct
EILDV

(19) United States
(12) Patent Application Publication
(10) Pub. No.: US 2008/0050812 A1
(43) Pub. Date: Feb. 28, 2008

(54) Micropositioning Cells For Tissue Engineering

(76) Inventors: James W. Cox, Cambridge, TX (US); Robert J. Klebe, Helotes, TX (US)

Correspondence Address: NEEDLE & ROSENBERG, P.C., SUITE 1000, 999 PEACHTREE STREET, ATLANTA, GA, 30309-3915, US

(21) Appl. No.: 10/558,916

(22) PCT Filed: May 28, 2004
(86) PCT No.: PCT/US04/16828

§ 371 Date: Feb. 5, 2007

Related U.S. Application Data
(60) Provisional application No. 60/474,574, filed on May 30, 2003.

(51) Int. Cl. C12N 5/02; C07K 16/00; C12N 5/00

(52) U.S. Cl. 435/346; 435/395; 530/388.1

(57) ABSTRACT

The present invention features methods for specifically micropositioning cells on a substratum. Cell-adhesive substances that bind to naturally-occurring or artificially-attached cell-surface moieties are deposited onto the substratum (e.g., using an inkjet printer), after which cells are incubated with the substratum such that they attach to the substratum via the cell-adhesive substances. The methods of the invention can be used to generate complex two- and three-dimensional patterns containing multiple cell types, e.g., for tissue engineering.

### FIELD OF THE INVENTION

This invention generally relates to methods for specifically micropositioning cells onto a substratum, e.g., for tissue engineering.

### BACKGROUND OF THE INVENTION

Present tissue engineering approaches allow the construction of simple, avascular tissues. For example, engineered cartilage can be constructed using chondrocytes alone, and engineered skin can be constructed using a layer of fibroblasts combined with a layer of keratinocytes. However, to construct functional equivalents of more complex tissues, it will be necessary to combine multiple cell types in the appropriate histological configurations. Therefore, future progress in tissue engineering will require approaches for micropositioning combinations of different types of cells into anatomically correct patterns.

One approach for constructing engineered tissues involves positioning the cells onto a substratum by first depositing, onto the substratum, a cell adhesion molecule or antibody that binds to an endogenously-produced molecule on the surface of the cells to be deposited onto the substratum. However, the ability to microposition a large number of cell types within a tissue construct is currently limited by the very small number of adhesion molecules and antibodies that are known to specifically bind to a given cell type and not cross-react with any other cell type. Moreover, not all cell types display sufficient levels of cell-surface molecules that can be recognized by cell adhesion molecules or antibodies that can be used in current cell micropositioning techniques. Accordingly, there is a need in the art for methodologies that allow specific micropositioning of a large number of different cell types onto a substratum in two- and three-dimensional patterns.

### SUMMARY OF THE INVENTION

The present invention provides methods for specifically micropositioning multiple types of cells onto a substratum.

In a first aspect, the invention features a method for positioning cells onto a substratum. The method includes the steps of: a) depositing onto the substratum an adapter antigen-binding molecule; and b) contacting the substratum with an adapter antigen-labeled cell, thereby positioning the cell onto the substratum.

The adapter antigen-binding molecule can be deposited onto the substratum in a pattern, e.g., using an inkjet printer.

In various embodiments of the first aspect of the invention, the adapter antigen-binding molecule can be, e.g., a polypeptide (e.g., an antibody, avidin, neutravidin or streptavidin), and the adapter antigen can be, e.g., a peptide, a polypeptide, biotin, a fluorophore, a small molecule (hapten), a pharmaceutical compound, or a carbohydrate.

In other embodiments of the first aspect of the invention, the adapter antigen-labeled cell can be labeled with two or more different adapter antigens.

In still other embodiments of the first aspect of the invention, at least two different adapter antigen-binding molecules can be deposited onto the substratum.

In yet other embodiments of the first aspect of the invention, at least two layers of cells can be deposited onto the substratum.

In a second aspect, the invention features a method of positioning cells onto a substratum. The method includes the steps of: a) depositing onto the substratum at least two different cell-adhesive substances; b) inactivating all but one of the cell-adhesive substances to provide one active cell-adhesive substance and at least one inactive cell-adhesive substance deposited onto the substratum; c) contacting the one active cell-adhesive substance with a cell, thereby affixing the cell to the substratum; d) re-activating an inactive cell-adhesion substance; and e) contacting the re-activated cell-adhesive substance with a cell, thereby affixing the cell to the substratum, thereby positioning cells onto the substratum.

The cell-adhesive substances can be deposited onto the substratum in a pattern, e.g., using an inkjet printer.

In various embodiments of the second aspect of the invention, unbound cells can be removed prior to re-activating an inactive cell-adhesion substance.

In other embodiments of the third aspect of the invention, at least three different cell-adhesive substances can-be deposited onto the substratum, and step d) can repeated to re-activate a third cell-adhesive substance, and step e) can be repeated to affix a cell to the substratum via the third cell-reactive substance.

In other embodiments of the second aspect of the invention, at least four different cell-adhesive substances can be deposited onto the substratum, and step d) can be repeated to re-activate a fourth cell-adhesive substance, and step e) can be repeated to affix a cell to the substratum via the fourth cell-reactive substance.

Cell-adhesive substances for use in the methods of the second aspect of the invention can be, e.g., cell adhesion molecules, lectins, or antibodies.

In a third aspect, the invention features a monoclonal antibody that specifically binds to Bodipy-FL, wherein the monoclonal antibody has the same binding specificity as the monoclonal antibody produced by hybridoma cell line BFL-F12. For example, the monoclonal antibody can be the monoclonal antibody produced by hybridoma cell line BFL-F12.

In a fourth aspect, the invention features a hybridoma cell line that produces a monoclonal antibody that specifically binds to Bodipy-FL, wherein the monoclonal antibody has the same binding specificity as the monoclonal antibody produced by hybridoma cell line BFL-F12. For example, the hybridoma cell line can be BFL-F12.

In a fifth aspect, the invention features a method of binding an antibody to Bodipy-FL, comprising contacting Bodipy-FL with a monoclonal antibody that specifically binds to Bodipy-FL, wherein the monoclonal antibody has the same binding specificity as the monoclonal antibody produced by hybridoma cell line BFL-F12, thereby binding the antibody to Bodipy-FL.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 is a diagram showing the strategy for micropositioning a layer of cells onto a single substratum. The cells that are labeled with an adapter antigen (small squares) are positioned using an antibody against the adapter antigen (Ab-v), which has been printed onto the substratum.

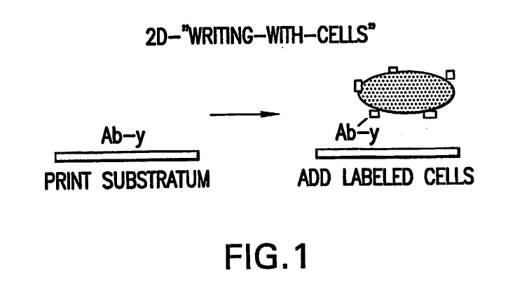

FIG. 2 is a diagram showing the strategy for micropositioning two or more layers of cells onto a single substratum using a first layer of cells that are labeled with two adapter antigens (light and dark small squares); the first layer is positioned using an antibody against the first adapter antigen (Ab-y), and the second layer of cells (labeled with the second adapter antigen (dark squares)) is positioned using an antibody against the second adapter antigen (Ab-r).

FIG. 3A-3C is a series of panels showing that adapter antigen-labeled cells bind specifically to subtrata labeled with the appropriate adapter antigen-binding molecule: Panel A: cells labeled with biotin attached only to the word “Avidin” (printed with neutravidin); Panel B: cells labeled with fluorescein attached only to the word “Antibody” (printed with an anti-fluorescein antibody); Panel C: two populations of cells, one labeled with fluorescein and one labeled with biotin, attach to the word “Antibody” or “Avidin” depending upon the adapter antigen label.

### DETAILED DESCRIPTION OF THE INVENTION

The materials, compositions, devices, and methods described herein may be understood more readily by reference to the following detailed description of specific embodiments of the disclosed subject matter, and methods and the Examples included therein and to the Figures and their previous and following description.

Before the present materials, compositions, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Disclosed herein are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide or nucleic acid is disclosed and a number of modifications that can be made to a number of amino acid residues or nucleotides, including those related to the cell adhesive substances, are discussed, each and every combination and permutation that are possible are specifically contemplated unless specifically indicated to the contrary. Thus, if a class of substituents A, B, and C are disclosed as well as a class of substituents D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

In this specification and in the claims that follow, reference will be made to a number of terms, which shall be defined to have the following meanings:

As used in the specification and the appended claims, the singular forms “a,” “an,” and “the” include plural referents unless the context clearly dictates otherwise. Thus, for example, reference to “a nucleotide” includes mixtures of two or more such nucleotides, reference to “an antibody” includes mixtures of two or more such antibodies, reference to “the cell” includes mixtures of two or more such cells, and the like.

“Optional” or “optionally” means that the subsequently described event or circumstance can or cannot occur, and that the description includes instances where the event or circumstance occurs and instances where it does not.

The ability to engineer complex tissues containing multiple cell types affixed to a substratum in a desired pattern has been limited by an insufficient number of known cell adhesion molecules and antibodies that bind to endogenous cell-surface molecules in a cell type-specific manner. The present invention overcomes this limitation by providing two new approaches for specifically micropositioning multiple cell types together on a substratum in any desired two- or three-dimensional pattern.

### Method One: The “Sequential” Micropositioning Method

In the sequential micropositioning method of the present invention, two or more different cell-adhesive substances (e.g., cell adhesion molecules) that bind to naturally occurring cell-surface molecules are deposited onto a substratum in a desired pattern. Cells can then be bound to the substratum via the deposited cell adhesion molecules. Two or more different types of cells can be specifically and sequentially micropositioned onto a single substratum using this method, by selectively inhibiting binding to all but the first type of adhesion molecule, saturating the first type of deposited adhesion molecule with the first type of cell to be deposited, and then sequentially re-activating each type of cell adhesion molecule in turn.

In one example, to specifically microposition four different types of cells using the sequential method, poly-L-lysine, fibronectin, concanavalin-A, and neutravidin can be deposited onto a substratum, each in its own pattern (e.g., using an inkjet printer or other suitable approach). Low-temperature incubation (4° C.), and mannose or methyl-α-D-mannopyranoside (50 mM at 37° C. for 15 min.) are used to inhibit, respectively, fibronectin and concanavalin-A. Biotinylation of cells (using 0.1 mg/ml of Sulfo-N-hydroxysuccinimide-biotin (Sulfo-NHS-biotin)) is required for cells to attach to neutravidin at 37° C. Cells will attach to poly-L-lysine under all conditions, and thus, cells that are to be attached to the deposited poly-L-lysine are added first. After all poly-L-lysine binding sites are saturated and the unbound cells are washed away, one of the two remaining types of cell adhesion molecules on the substratum is re-activated, after which the substratum is incubated with a saturating number of cells belonging to the second cell type to be deposited, and unbound cells are washed away. The process is repeated for the third type of cell adhesion molecule and cell type, as described in the Examples below. Lastly, cells labeled with biotin are added and allowed to attach to areas coated with neutravidin.

### Cell-Adhesive Substances for Sequential Micropositioning

The cell-adhesive substances for use in the sequential micropositioning methods of the present invention can be, e.g., cell adhesion molecules, which are known to bind to molecules that are naturally found on cell surfaces or that can be attached to cell surfaces (e.g., biotin), thereby allowing cells to adhere to surfaces upon which such cell adhesion molecules are found. Numerous such cell adhesion molecules are known in the art. For example, fibronectin is a well-known cell adhesion molecule that can be used in the sequential micropositioning methods of the invention, as described herein. Other examples of cell adhesion molecules that can be used in the methods of the invention include (but are not limited to): avidin, streptavidin, neutravidin, laminin, vitronectin, chondronectin, epinectin, epibolin, uromorulin, merosin, collagen, fibrinogen, L-CAM, N-CAM, VCAM-1, thrombospondin, and selectin. The above cell adhesion molecules can, in some cases, be inhibited by low temperature, lack of divalent cations, or inhibitory peptides, as shown in Table 1.

TABLE 1

Examples of Cell Adhesion Proteins and their
Inhibitory Peptides.

| | |
|---|---|
| Cell Adhesion | Inhibitory |
| Protein | Peptide |

| | | |
|---|---|---|
| Fibronectin | RGD | SEQ ID NO: 1 |
| Collagen | DGEA | SEQ ID NO: 2 |
| Laminin | YIGSR | SEQ ID NO: 3 |
| Fibrinogen | GPRP | SEQ ID NO: 4 |
| Thrombospondin | KQAGDV | SEQ ID NO: 5 |
| VCAM-1 | EILDV | SEQ ID NO: 6 |

(Reference: Yamada, K.M. Adhesive Recognition Sequences. J. Biol. Chem. 266:12809-12812, 1991)

Lectins, such as concanavalin A, wheat germ agglutinin, etc., can be inhibited by specific mono- and disaccharides, as is well-understood by those of ordinary skill in the art. Avidin, streptavidin, and neutravidin can be inhibited by biotin and derivatives of biotin. Poly-L-lysine and other highly charged polymers can be used as adhesive substances as well, but must be used as the first step because their adhesive activity cannot be selectively inhibited and re-activated.

In addition to the above-mentioned cell-adhesive substances, other adhesive materials (e.g., but not limited to, antibodies) that bind to naturally occurring cell-surface molecules or to molecules that can be attached to cell surfaces (e.g., as described herein below) can be used in the sequential micropositioning methods of the invention.

### Method Two: The “Adapter Antigen” (Simultaneous) Micropositioning Method

In the adapter antigen micropositioning method of the present invention, a foreign antigen (“adapter antigen”), e.g., a fluorophore, is coupled to the surface of the cells to be micropositioned, as described below. An antibody (“adapter antibody”) or other molecule (“adapter antigen-binding molecule”) that specifically binds to the adapter antigen is deposited onto a substratum in a desired pattern (e.g., using an inkjet printer, plotter, or other suitable approach), after which the adapter antigen-modified cells are incubated with the modified substratum. The modified cells attach to the substratum via the immobilized antibodies or adapter antigen-binding molecules.

Virtually any cell type can be specifically micropositioned using the adapter antigen approach. Moreover, using a different antigen-antibody pair for each cell type to be incorporated into a cell layer, many different cell types can be simultaneously and selectively micropositioned on a substratum, thereby facilitating the construction of complex tissue types.

The use of adapter antigens for cell micropositioning has numerous advantages over the use of cell type-specific monoclonal antibodies that bind to antigens that are naturally found at the cell surface. Although cell type-specific monoclonal antibodies that bind to naturally occurring cell-surface antigens can be used to microposition cells (assuming that the cell type of interest possesses one or more such cell type-specific surface antigens), useful antibodies against such antigens are often difficult to isolate. For example, a cell type-specific monoclonal antibody may have an insufficient binding constant or may recognize a cell-surface antigen that is present at an insufficient level to allow adequate anchorage of the cell type of interest onto the substrate. In addition, a cell-surface antigen recognized by a cell type-specific antibody might vary in its expression when the target cell type is placed in culture, or might depend on unknown factors for its expression. By contrast, the labeling of cells with a foreign adapter antigen results in both high specificity of antibody binding and the ability to experimentally control the cell surface density of such adapter antigens.

Another important advantage of the adapter antibody method is that this approach can be used to construct three-dimensional tissues. As described below, a given cell type can be labeled with more than one adapter antigen, which can be used to govern the attachment of that cell type to any other cell type (by contrast, an antibody against a naturally occurring cell type cannot be used to link together different cell types, since such an antibody will only recognize one type of cell).

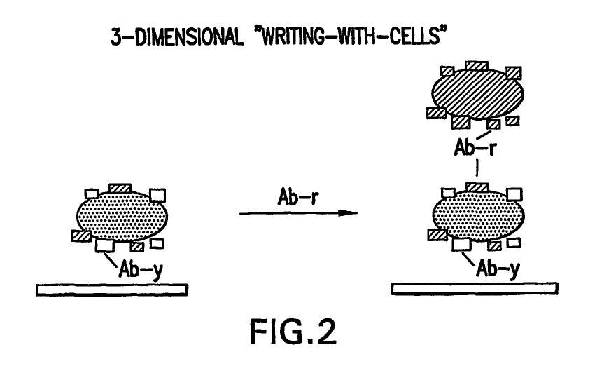

Yet another important advantage of the adapter antibody method is that this approach can be used to construct three-dimensional tissues. By contrast, an antibody against a naturally occurring cell type-specific antibody cannot be used to link together different cell types, since such an antibody will only recognize one type of cell. For example, cells can be labeled with two adapter antigens, one of which is used to attach the cells to a substratum using an antibody that specifically recognizes this first adapter antigen. A second layer of cells can then be constructed upon the first layer, by incubating the first layer of cells with an antibody that specifically binds the second adapter antigen, after which the second cell type (labeled with the second adapter antigen) is added. Because antibodies are multivalent, the second antibody acts as a bridge to affix the second layer of cells to the first (FIG. 2). The cells in the second layer can be labeled with more than one adapter antigen, such that a third cell layer (e.g., labeled with the first adapter antigen or with a third adapter antigen) can be added, and so on.

By using two adapter antigens per cell, it is possible to construct three-dimensional tissues containing multiple cell types. By having several adapter antigen/adapter antibody pairs, one can construct complex tissues containing multiple cell types arranged in any desired anatomical pattern. Layers of cells containing different cell types can be formed as follows. For example, the first layer of cells can contain cells labeled with antigens A and B, wherein a substratum-bound antibody against antigen A is used to attach the double-labeled cells to the substratum. An antibody against antigen B is then incubated with and allowed to bind to the cell layer, after which cells that are double-labeled with antigens B and C are added, and form a second layer by virtue of their attachment to the antibody against antigen B. A third layer can be constructed by adding an antibody against antigen C, after which cells that are labeled with antigen C are bound to antibody C. Cells in the third layer are double-labeled, e.g., with antigen C plus antigen D (or antigen A or B) if more than three layers are desired, and so on. In this fashion, very complex tissues can be assembled layer by layer. Placing one ring of cells on top of another ring of cells is an iterative process that can be used to form a tube (e.g., a blood vessel). Double-labeling with adapter antigens can thus be used to construct tissues in any desired pattern.

### Deposition of Cell-Adhesive Substances onto Substrata

Cell-adhesive substances for use in the micropositioning methods of the invention can be deposited onto substrata in specific patterns using, e.g., inkjet printers or analogous approaches. By “cell-adhesive substance” is meant any molecule that can be used in either the sequential or simultaneous (adapter antigen-mediated) methods of the invention to affix a cell to a substratum. Cell-adhesive substances include, e.g., cell adhesion molecules, and adapter antigen-binding molecules (e.g., an antibody or other molecule (e.g., neutravidin or streptavidin) that specifically binds to a particular adapter antigen and does not appreciably bind to other molecules).

Inkjet printing technology has advanced to a stage that now allows photographic quality digital printing. Standard inkjet printers can deposit millions of droplets with a precision of a cell diameter within seconds; specifically, resolutions of approximately twenty micrometers are achievable using inexpensive office inkjet printers that can print an entire 8.5×11 inch page in about four seconds (see, e.g., www.epson.com/cgi-bin/Store/index.jsp), which is an industry standard for text printing. Moreover, the current technology allows individual control of the volume of an ink droplet, such that droplet size can be varied between two and six picoliters (see, e.g., www.epson.com/cgi-bin/Store/index.jsp). Current inkjet printing technology permits the simultaneous deposition of droplets of several colors of ink while maintaining high precision in their placement. For example, standard office inkjet printers can deposit four colors of ink simultaneously, photographic quality inkjet printers can deposit six different inks simultaneously, and high-end printers deposit up to sixteen different inks in a single printing event (www.inkjetart.com). One of ordinary skill in the art will appreciate that printers having the ability to deposit any number of different types of ink can also be used in the methods of the invention.

Both piezoelectric (e.g., but not limited to, those produced by Epson, Inc.) and bubblejet (e.g., but not limited to, those produced by Hewlett-Packard Co. or Canon, Inc.) inkjet printers can be used to deposit cell-adhesive substances (e.g., cell adhesion proteins, adapter antibodies, and/or other adapter antigen-binding molecules (e.g., streptavidin or neutravidin)) onto substrata. The type of inkjet printer to be used in the methods of the invention is not critical, as long as it can print the desired cell-adhesive substances onto the substrate in the desired pattern with the desired resolution, such that the cell-adhesive substances being printed retain the ability to bind to their intended cell-surface target. See, e.g., U.S. Pat. No. 5,108,926, herein incorporated by reference in its entirety, for a description of the use of an inkjet printer to deposit cell adhesion materials onto substrata in specific patterns.

Substrata

Substrata for use in the methods of the invention can be any material onto which cell-adhesive substances and cells can be deposited, and which will be non-toxic to the deposited cells, and non-toxic to any intended recipient (e.g., a patient or subject) of such an engineered tissue construct, as will be understood by one of ordinary skill in the art. For a tissue construct that will not be implanted into a recipient, any material that is non-toxic to the deposited cells can be used as a substratum in the methods of the invention, including (but are not limited to): acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, TEFLON, fluorocarbons, nylon, polyorthoesters, functionalized silane, and polypropylfumerate.

For a tissue construct that can be implanted into a recipient, any material that is non-toxic to the deposited cells and non-toxic to the recipient can be used as a substratum in the methods of the invention, including (but are not limited to) silicon rubber, silicone, polyanhydrides, polyglycolic acid, polylactic acid, collagen, gelatin, glycosaminoglycans, polyamino acids, polyhydroxyalkanoate, polyglycolic acid (PGA), polylactic acid (PLA), Polyglactin 910 (comprising a 9:1 ratio of glycolide per lactide unit, also known as VICRYL), polyglyconate (comprising a 9:1 ratio of glycolide per trimethylene carbonate unit, also known as MAXON), and polydioxanone (PDS) (see, e.g., U.S. Pat. No. 6,514,515), polylactide/dextran co-polymers (see, e.g., U.S. Pat. No. 6,525,145), fibrin (see, e.g., U.S. Pat. No. 6,331,422), as well as any other non-toxic material that is suitable for implanting into recipient, e.g., any biodegradable polymer intended for such purpose. Of course, materials that are suitable for implanting into recipients can also be used as substrata in constructs that are not intended for implantation into recipients.

Substrata can be porous or non-porous. The skilled artisan will understand that the substrata can have any useful form or shape, including, but not limited to, thin film, membrane, sheet, or chip.

### Cells for Micropositioning

It will be apparent to one of ordinary skill in the art that any type of cell can be micropositioned using the methods of the invention, as long as: 1) the surface of the cell displays at least one endogenously-produced molecule that can be bound by a cell-adhesive substance that is suitable for use in the sequential methods of the invention, or 2) the cell can be modified by attaching an adapter antigen to its cell surface. Cells that can be used in the methods of the invention include primary cells, e.g., those derived directly from blood, body tissue, or organs. Cultured primary cells or cells from established cell lines can also be used in the methods of the invention. For example, any type of stem cell or other undifferentiated cell (e.g., from a patient, donor, embryo, cell line, or other source) can be differentiated into the appropriate cell type in vitro, as will be understood by those of ordinary skill in the art, and then micropositioned using the methods described herein. Alternatively, undifferentiated cells can be micropositioned, after which the cells are allowed to differentiate in situ (e.g., by interacting with other micropositioned cells in the engineered tissue or by treating the engineered tissue with the appropriate cytokines or other differentiation-inducing stimuli).

Cells used in the methods of the invention can be autologous, heterologous, syngeneic, or xenogeneic. Those of ordinary skill in the art will understand how to choose the appropriate source of cells, depending upon the ultimate use for the tissue construct. For example, in some cases it might be most desirable to use a patient's own cells to avoid evoking an immune response against a tissue construct to be implanted within the patient. Moreover, as is known in the art, animals can be engineered to lack immunogenic cell surface molecules (see, e.g., Phelps et al. “Production of alpha 1,3-galactosyltransferase-deficient pigs.” Science 299:411-414 2003); therefore, in some cases it may be appropriate or desirable to use cells from xenogeneic sources (e.g., but not limited to, mice, pigs, sheep, goats) in the methods of the invention, or human cells in which the necessary histocompatibility genes have been knocked out to achieve histocompatibility with the intended recipient of the tissue construct. One of skill in the art will understand that in constructs to be used in vitro (e.g., to study cell-cell interactions and/or cell behavior), issues of histocompatibility need not be considered. Examples of cells that can be micropositioned using the methods of the invention include, but are not limited to: fibroblasts, keratinocytes, chondroblasts, chondrocytes, osteoblasts, osteocytes, endothelial cells, epithelial cells, melanocytes, embryonic stem cells, epidermal stem cells, CD34 lymphoid stem cells, neurons, glial cells, microglia, astrocytes, bone marrow cells, muscle cells (e.g., skeletal myoblasts, cardiac myoblasts, satellite cells), pancreatic cells (e.g., beta-cells), liver cells (e.g., hepatocytes), hematopoietic cells, and kidney cells.

### Adapter Antigens

An adapter antigen, as defined herein, is any foreign molecule that can be covalently or non-covalently attached to the surface of a cell, and thereby used to affix the cell onto a substratum via an antibody or other molecule adapter antigen-binding molecule. A “foreign” molecule, in this context, is a molecule that is not normally produced by the cell to which the adapter antigen is to be attached; since molecules that are found in the body can be used to elicit antibodies (e.g., hormones such as insulin, L-thyroxine, aldosterone, or peptides derived from endogenous proteins), adapter antigens can be molecules that are not foreign to the body. Any molecule to be used as an adapter antigen should be attachable to the cell under conditions that are non-toxic to the cell. Furthermore, the adapter antigen itself should be non-toxic to the cell to which it is attached.

The adapter antigen must be capable of being specifically recognized and bound by an antibody or other adapter antigen-binding molecule. Suitable adapter antigens are readily commercially available and/or can be synthesized by those of ordinary skill in the art. The particular adapter antigens to be used in accordance with the present invention can be chosen by one of ordinary skill in the art based on factors such as cost, convenience, availability, compatibility with various conditions, and the like.

Examples of suitable adapter antigens that can be covalently or non-covalently attached to cells include (but not limited to): biotin, fluorescent dyes, natural or synthetic organic or inorganic compounds (e.g., haptens as described below), pharmaceutical compounds, vitamins, carbohydrates (e.g., A, B or other blood group antigens, or other naturally occurring or synthetic polysaccharides), and naturally occurring or artificial polypeptides or peptides (as long as the antibody or molecule that specifically binds a chosen polypeptide or peptide adapter antigen does not cross-react with an endogenous cell-surface molecule). Any length of peptide (e.g., but not limited to, at least 4, 5, 6, 7, 8, 9, or 10 amino acids, or more than 10 amino acids) is suitable for an adapter antigen, as long as the peptide can be used to raise a peptide-specific antibody that will not cross-react with an endogenous cell-surface molecule. Peptide adapter antigens can be linear or cyclic peptides, and can include peptides having modified amino acids, and antibiotics (for example, but not limited to, beta-lactams and defensins). For in vivo applications (e.g., tissue constructs that are to be implanted into patients or subjects), molecules that are non-toxic, biodegradable, and/or readily secreted are preferred.

Examples of haptens, hormones, antibiotics, and pharmaceutical compounds that can be used as covalently or non-covalently attached adapter antigens in the methods of the invention include, e.g. (but are not limited to): penicillin, polymyxin, thyroxine, probenecid, carboxybenzoxyglycyl-L-phenylalanine, hippuryl-L-phenylalanine, N-ethylmaleimide, or N-(4-hydroxy-1-naphyl)-maleimide, dinitrophenol, estrogen, progesterone, cortisone-21-hemisuccinate, cholic acid, thyroxine, prostaglandins, reserpine, clonazepan-3-hemisuccinate, poly(DL-alanyl)-poly(L-lysine), tobramycin, cocaine, digitoxigenin, gentamicin, chloramphenicol, gentamicin, adriamycin, and so on (see, e.g., B. F. Erlanger, “The preparation of antigenic hapten-carrier conjugates: A survey,” Methods in Enzymology 70: 85-104, 1980).

A free (i.e., not bound to a cell) adapter antigen must be capable of being bound to the cell surface. Therefore, if a covalent attachment is desired and the adapter antigen does not contain a chemically reactive moiety that can be used to link the adapter antigen to a cell, the adapter antigen must be derivatized with such a linking moiety. The linking moiety can be directly connected to the adapter antigen or the linking moiety can be connected to the adapter antigen via a spacer moiety (e.g., but not limited to, an alkyl, ester, ether, or amide-containing chain). Suitable adapter antigens that are derivatized with linking moieties are readily commercially available and/or can be synthesized by those of ordinary skill in the art. The particular derivatized adapter antigens that can be used in accordance with this invention can be chosen by one of ordinary skill in the art based on factors such as cost, convenience, availability, compatibility with various reaction conditions, the types of cells being used, and the like.

Alternatively, the adapter antigen and/or the linking moiety can be purchased from commercial sources separately and/or they can be prepared separately by synthetic methods known in the art. If obtained separately, the adapter antigen and the linking moiety can be brought together and either directly connected or connected with a spacer moiety between them by synthetic methods known in the art of organic synthesis.

Non-covalent bonding of adapter antigens to the cell surface can also be carried out by the use of antibodies to cell surface molecules, lectins, or by intercalation of lipophilic moieties into the cell membrane lipid bilayer. Examples of non-covalently attached adapter antigens that can be used in the methods of the invention include:

a) Membrane Intercalating Agents:
i. Detergents and lipofection reagents: These compounds are water soluble (or form small micelles) and will integrate into membranes, as is well understood by those of ordinary skill in the art.
ii. Antigens added by the liposome fusion method: Briefly, liposomes are prepared by drying a lipid in a glass vessel with nitrogen. Next, an aqueous buffer is added and the solution is sonicated to form liposomes. The liposomes are then allowed to fuse with cells and thereby integrate their contents into the cell surface. (See, e.g., Zho, F, Neutra, MR, “Antigen delivery to mucosa-associated lymphoid tissues using liposomes as a carrier,” Biosci. Rep. 22:355-369, 2002).

b) Lectins or Antibodies to Natural Cell Surface Components:
Lectins or antibodies that bind to natural cell surface components can be derivatized with a small molecule (a hapten), a fluorophore, or another adapter antigen (i.e., a molecule that can be recognized by an adapter antigen-binding molecule); the antibodies are used to non-covalently attach such adapter antigens to cells.

### Linking Moiety

The linking (reactive) moiety allows the adapter antigen to react with and form a chemical bond with a substituent on a cell surface. The reaction between the linking moiety and the cell-surface substituent results in a chemical bond that links the adapter antigen to the cell surface. Such reactions can occur as a result of a direct nucleophilic or electrophilic interaction between the linking moiety and the cell-surface substituent. For example, a nucleophilic linking moiety can directly react with an electrophilic cell-surface substituent and form a bond that links the adapter antigen to the cell surface. Alternatively, an electrophilic lining moiety can directly react with a nucleophilic cell-surface substituent and form a bond that links the adapter antigen to the cell surface. Also, the adapter antigen can be covalently attached to a cell surface by an indirect interaction where a reagent initiates, mediates, or facilitates the reaction between the linking moiety of the adapter antigen and the cell-surface substituent. For example, the bond-forming reaction between the linking moiety and a cell-surface substituent can be facilitated by the use of a coupling reagent (e.g., carbodiimides, which are used in carbodiimide-mediated couplings) or enzymes (e.g., glutamine transferase).

As noted above, suitable linking moieties are readily commercially available and/or can be synthesized by those of ordinary skill in the art. And the particular linking moieties that can be used in accordance with this invention can be chosen by one of ordinary skill in the art based on factors such as cost, convenience, availability, compatibility with various reaction conditions, the type of cell-surface substituent with which the linking moiety is to interact, and the like.

For example, Molecular Probes (Eugene, Oreg.) manufactures a wide variety of water-soluble amine-reactive fluorescent dyes, biotins, and other haptens for conjugation to proteins and other amine-containing compounds. These haptens have an amine-reactive 4-sulfo-2,3,5,6-tetrafluorophenyl (STP) ester group and can be used to label cells with adapter antigens in the absence of organic solvents. Pierce Biotechnology, Inc. (Rockford, Ill.) also manufactures a wide variety of N-hydroxylsuccinimide (NHS)-derivatized and N-hydroxysulfosuccinimide (sulfo-NHS)-derivatized haptens (e.g., biotin and fluorescent dyes) that can be used to label cells with adapter antigens in the absence of organic solvents.

### Electrophilic Linkers and Nucleophilic Cell-Surface Substituents

The adapter antigen can be derivatized with a linking moiety that can directly or indirectly react with a nucleophilic cell-surface substituent and form a chemical bond.

A nucleophilic cell-surface substituent is a molecule or compound on the surface of the cell with a nucleophilic or potentially nucleophilic functional group, i.e., a functional group with an electron-rich atom. Examples of such nucleophilic cell-surface substituents that can react with and form a bond to a linking moiety include, but are not limited to, proteins, peptides, or receptors that possess amino acid residues with a nucleophilic or potentially nucleophilic amine, carboxylate or carboxylic acid, alcohol, or thiol functional group (e.g., cysteine, serine, threonine, tryptophan, tyrosine, aspartic acid, glutamic acid, glutamine, arginine, histidine, and lysine). Other examples of nucleophilic cell-surface substituents include, but are not limited to, carbohydrates, polysaccharides, lipids, saturated and unsaturated fatty acids, sphingolipids, or cholesterols that possess a nucleophilic or potentially nucleophilic amine, carboxylate, alcohol, or thiol functional group.

Further, it is contemplated that more than one type of nucleophilic cell-surface substituent will be present on a cell surface and, as such, they can be selectively reacted with the linking moiety on the adapter antigen. For example, a cell-surface peptide with both nucleophilic amine and carboxylate functional groups can be treated with alkylating agents to block the amine functional groups and leave the carboxylate groups available to react with the linking moiety of the adapter antigen. Conversely, by controlling the reaction conditions (e.g., temperature and concentration) the more reactive amine group can be selectively reacted with the linking moiety and leave the less reactive carboxylate groups mostly unreacted.

When the adapter antigen is to be attached to a cell surface using nucleophilic cell-surface substituents, such as those listed above, the linking moiety of the adapter antigen is typically electrophilic or potentially electrophilic, i.e., it contains an electron-deficient atom. Examples of such electrophilic linking moieties include, but are not limited to, aldehydes, acyl derivatives (e.g., acyl azides, acyl nitriles), esters and activated esters (e.g., succinimidyl esters, sulfosuccinimidyl esters), anhydrides and mixed anhydrides, derivatized carboxylic acids and carboxylates, imines, isocyanates, isothiocyanates, sulfonyl chlorides, organo-halides, and maleimides. These moieties are well known in the art of organic chemistry.

Also, when a linking moiety on the adapter antigen is not generally reactive it can be converted into more reactive linking moieties. For example, linking moieties that contain carboxylate or carboxylic acid groups may, depending on the conditions, not be very reactive toward a nucleophilic cell-surface substituent. However, these linking moieties can be converted into more reactive, activated esters by a carbodiimide coupling with a suitable alcohol, e.g., 4-Sulfo-2,3,5,6-tetrafluorophenol, N-hydroxysuccinimide or N-hydroxysulfosuccinimide. This results in a more reactive, water-soluble activated ester linking moiety.

When the nucleophilic cell-surface substituent contains an amine functional group, it can be particularly reactive toward adapter antigens with electrophilic linking moieties. Such amine containing cell-surface substituents can react with the linking moiety of the adapter antigen and form, for example, depending on the linking moiety, amine, amide, carboxamide, sulfonamide, urea, or thiourea bonds. When the nucleophilic cell-surface substituent contains a carboxylate, they can react with the linking moiety of the adapter antigen and form, for example, depending on the linking moiety, esters, thioesters, carbonates, or mixed anhydrides. When the nucleophilic cell-surface substituent contains an alcohol or thiol, they can react with the linking moiety of the adapter antigen and form, for example, depending on the linking moiety, esters, thioesters, ethers, sulfides, disulfides, carbonates, or urethanes.

The kinetics of such reactions depends on the reactivity and concentration of both the adapter antigen and the nucleophilic cell-surface substituent. Also, buffers that contain free amines such as Tris and glycine should be avoided when the adapter antigen is to be attached through a nucleophilic cell-surface substituent. In addition, high concentrations of nucleophilic thiols should be avoided because they may compete with the cell-surface substituent for the linking moiety of the adapter antigen.

Also, significant factors affecting the reactivity of a cell-surface substituent with an amine functional group are the amine's class and basicity. For example, many proteins have lysine residues, and most have a free amine at the N-terminus. Aliphatic amines, such as the amino group of lysine, are moderately basic and reactive with most electrophilic linking moieties. However, the concentration of the free base form of aliphatic amines below pH 8 is low; thus, the kinetics of a reaction between an aliphatic amine on the cell-surface and, for example, an isothiocyanates or succinimidyl ester linking moiety can be strongly pH dependent. While a pH of 8.5 to 9.5 is most efficient for attaching an adapter antigen with an electrophilic linking moiety to a cell's surface by a cell-surface lysine residue, there will be some reactivity at pH 7 to pH 8, and reactions are not generally carried out much above pH 8, as this would be toxic to cells. In contrast, the amino group at the N-terminus of a protein usually has a pKₐ of ˜7, so it can sometimes be selectively modified by reaction at near neutral pH.

As noted above, the nucleophilic cell-surface substituents react directly or indirectly with electrophilic linking moieties on the adapter antigen. For example, nucleophilic cell-surface substituents will react with isocyanate linking moieties. Isocyanate linking moieties are readily derivable from acyl azide linking moieties, and they react with cell-surface substituents that contain amine functional groups to form ureas, they react with cell-surface substituents that contain alcohols to form urethanes, and they react with cell-surface substituents that contain thiols to form thiourethanes.

Isothiocyanate linking moieties are an alternative to isocyanates and are moderately reactive but quite stable in water. Isothiocyanate linking moieties with react with an amine, alcohol, or thiol containing cell-surface substituents to form thioureas, and thiourethanes.

Succinimidyl ester linking moieties can also react with cell-surface substituents that contain amine, carboxylate, alcohol, or thiol functional groups. Succinimidyl ester linking moieties are particularly reactive towards amines, where the resulting amide bond that is formed is as stable as a peptide bond. However, some succinimidyl ester linking moieties may not be compatible with a specific application because they can be quite insoluble in aqueous solution. To overcome this limitation, sulfosuccinimidyl ester linking moieties, which typically have higher water solubility than succinimidyl ester linking moieties, can be used. Sulfosuccinimidyl ester linking moieties can generally be prepared in situ from simple carboxylic acid linking moieties by dissolving an adapter antigen with a carboxylic acid linking moiety in an amine-free buffer that contains N-hydroxysulfosuccinimide and 1-ethyl-3-(3-dimethylaminopropyl)carbodiimide. Also, 4-Sulfo-2,3,5,6-Tetrafluorophenol (STP) ester linking moieties can be prepared from 4-sulfo-2,3,5,6-tetrafluorophenol in the same way as sulfosuccinimidyl ester linking moieties. Sulfosuccinimidyl-esters and STP esters have the additional advantage that they will not penetrate the cell membrane due to the charged sulfate group and, thus, only labeling of cell surface components will occur.

As noted above, carboxylic acid linking moieties can be converted into more highly reactive linking moieties. For example, a carboxylic acid linking moiety can be converted into an activated ester or mixed anhydride, which can be used to modify less reactive aromatic amines and alcohol containing cell-surface substituents.

Sulfonyl chloride linking moieties are highly reactive but these reagents can be unstable in water, especially at the higher pH required for reaction with some aliphatic amines. Accordingly, attaching adapter antigens with sulfonyl chloride linking moieties to nucleophilic cell surface substituents is best done at low temperatures. If the nucleophilic cell-surface substituent contains an amine, the sulfonamide bond that is formed is extremely stable. Further, sulfonyl chloride linking moieties can also react with phenols (including tyrosine), aliphatic alcohols (including polysaccharides), thiols (such as cysteine) and imidazoles (such as histidine).

Aldehyde linking moieties will react with nucleophilic cell-surface substituents that contain amines to form Schiff bases.

Organo-halide linking moieties contain a carbon atom bonded to a halide (e.g., fluorine, chlorine, bromine, or idodine). These moieties will react with cell-surface substituents that contain amine, carboxylate, alcohol, thiol functional group to form, for example, amine, ester, ether, or sulfide bonds.

Suitable adapter antigens that contain electrophilic linking moieties that are capable of reacting directly or indirectly with a nucleophilic cell-surface substituent are commercially available from, for example, Molecular Probes (Eugene, Oreg.). Specific examples of such adapter antigens include, but are not limited to: 1-(3-(succinimidyloxycarbonyl)benzyl)-4-(5-(4-methoxyphenyl)oxazol-2-yl) pyridinium bromide, 1-(2-maleimidylethyl)-4-(5-(4-methoxyphenyl)oxazol-2-yl)pyridinium methanesulfonate, 2-(2,3-naphthalimino)ethyl trifluoromethanesulfonate, N-((2-(iodoacetoxy)ethyl)-N-methyl)amino-7-nitrobenz-2-oxa-1,3-diazole, 4-chloro-7-nitrobenzofurazan, 4-fluoro-7-nitrobenzofurazan, Alexa Fluor maleimides (e.g., Alexa Fluor. 488 C₅ maleimide and Alexa Fluor 594 C₅ maleimide), benz-2-oxa-1,3-diazoles, BODIPY-FL-STP, BODIPY iodoacetamides (e.g., BODIPY 507/545 iodoacetamide, BODIPY 530/550 iodoacetamide, BODIPY TMR cadaverine iodoacetamide), BODIPY maleimide, BODIPY methyl bromides (e.g., BODIPY 493/503 methyl bromide, BODIPY 630/650 methyl bromide), m-dansylaminophenylboronic acid, N,N′-dimethyl-N-(iodoacetyl)-N′-(7-nitrobenz-2-oxa-1,3-diazol-4-yl)ethylenediamine, eosin iodoacetamide, eosin maleimide, N-iodoacetyl(piperazinyl)sulfonerhodamine, N-methylisatoic anhydride, Oregon Green 488 (2′,7′-difluorofluorescein), carboxylic acid and succinimidyl ester derivatives of Oregon Green 488 isothiocyanate, Oregon Green 488 iodoacetamide, Oregon Green 488 maleimide, Rhodamine Red maleimide, tetramethylrhodamine iodoacetamide, tetramethylrhodamine maleimide, bromoacetamide and maleimide derivatives of Texas Red fluorophore, 7-fluorobenz-2-oxa-1,3-diazole-4-sulfonamide, Lucifer yellow iodoacetamide, maleimide and iodoacetamide derivatives of stilbene, QSY 7 maleimide, N-ethylmaleimide, N-(4-dimethylamino-3,5-dinitrophenyl)-maleimide, N-(4-hydroxyl-1-naphthyl)-isomaleimide, N-(4-hydroxyl-1-naphthyl)-maleimide, QSY 35 iodoacetamide, fluorescein-5-dichlorotriazine, fluorescein-5-iodoacetamide, fluorescein-5-maleimide, iodoacetamidofluorescein, fluorescein isothiocyanate, maleimidylpropionyl biocytin, Rhodamine Green, Rhodamine Red-X, tetramethylrhodamine isothiocyanate, Cascade Blue, Cascade Yellow, Marina Blue, Pacific Blue and AMCA-X fluorophores, fluorescamine, 3-(2-furoyl)quinoline-2-carboxaldehyde, 3-(4-carboxybenzoyl)quinoline-2-carboxaldehyde, o-phthaldialdehyde, naphthalenedicarboxaldehyde, 3-acylquinolinecarboxaldehyde, and dansyl chloride, Lissamine rhodamine B, Texas Red sulfonyl chloride, Texas Red-STP, ω-chloroacetophenone, Thyroxine, carbobenzoxy glycine-L-phenylalanine, benzoylglycyl-L-phenylalanine, chaulmoogric acid, cholanic acid, chlorophyll a, chlorophyllide a, and desthiobiotin.

### Nucleophilic Linkers and Electrophilic Cell-Surface Substituents

In another example, the adapter antigen can be derivatized with a linking that can react with an electrophilic cell-surface substituent and form a chemical bond.

An electrophilic cell-surface substituent is a molecule or compound on the surface of the cell with an electrophilic or potentially electrophilic functional group, i.e., a functional group with an electron-deficient atom. Examples of such electrophilic cell-surface substituents that can react with and form a bond to the linking moiety include, but are not limited to, proteins, peptides, or receptors that possess an electrophilic or potentially electrophilic atom (e.g., a carbonyl carbon atom, such as those found in esters and activated esters (e.g., succinimidyl esters, sulfosuccinimidyl esters), aldehydes, acyl derivatives (e.g., acyl azides, acyl nitriles), anhydrides and mixed anhydrides, or carboxylates, the carbon atom in an imine, isocyanates, or isothiocyanates, or halogenated carbon atoms). Other examples of electrophilic cell-surface substituents include, but are not limited to, carbohydrates, polysaccharides, lipids, saturated and unsaturated fatty acids, or cholesterols that possess an electrophilic or potentially electrophilic carbon atom such as those noted above.

Also, when an electrophilic cell-surface substituent is not generally reactive they can be converted into more reactive electrophilic cell-surface substituents. For example, cell-surface substituents that contain carboxylate or carboxylic acid groups may, depending on the conditions, not be very reactive toward a nucleophilic linking moiety. However, these cell-surface substituents can be converted into more reactive, activated esters by a carbodiimide coupling with a suitable alcohol, e.g., 4-sulfo-2,3,5,6-tetrafluorophenol or N-hydroxysulfosuccinimide. This results in a more reactive, electrophilic activated ester cell-surface substituent.

When the adapter antigen is to be attached to a cell surface with an electrophilic cell-surface substituent, such as those discussed above, the linking moiety of the adapter antigen is typically nucleophilic or potentially nucleophilic, i.e., the linking moiety contains an electron rich atom. Examples of suitable nucleophilic linking moieties include, but are not limited to, hydrazines, amines, alcohols, carboxylates, and thiols. These moieties are generally well known in the art of organic chemistry.

When the nucleophilic linking moiety contains and amine functional group, it can be particularly reactive toward electrophilic cell-surface substituents. Such amine containing linking moieties can react with the cell-surface substituent and form, for example, depending on the cell-surface substituent, amide, carboxamide, sulfonamide, urea, or thiourea bonds. When the nucleophilic linking moiety contains a carboxylate, they can react with the cell-surface substituent and form, for example, depending on the cell-surface substituent, esters, thioesters, carbonates, or mixed anhydrides. When the nucleophilic linking moiety contains an alcohol or thiol, they can react with the cell-surface substituent and form, for example, depending on the cell-surface substituent, esters, thioesters, ethers, sulfides, disulfides, carbonates, or urethanes.

As with the nucleophilic cell-surface substituent and electrophilic linking moiety interaction discussed above, the kinetics of the electrophilic cell-surface substituent and nucleophilic linking moiety reactions depends on the reactivity and concentration of both the adapter antigen and the cell-surface substituent. Also, buffers that contain free amines such as Tris and glycine should be avoided when the adapter antigen is to be attached through an electrophilic cell-surface substituent. In addition, high concentrations of nucleophilic thiols should be avoided because they may compete with the linking moiety for the cell-surface substituent of the adapter antigen.

Suitable adapter antigens with nucleophilic linking moieties capable of reacting directly or indirectly with an electrophilic cell-surface substituent are commercially available from, for example, Molecular Probes (Eugene, Oreg.). Specific examples of such adapter antigens include, but are not limited to, fluorescein-5-dichlorotriazine, Rhodamine, fluorescamine, Thyroxine, carbobenzoxy glycine-L-phenylalanine, benzoylglycyl-L-phenylalanine, chaulmoogric acid, cholanic acid, chlorophyll a, chlorophyllide a, 2-aminoacridone, 5-aminoeosin, 5-(aminoacetamido)fluorescein, 7-aminonaphthalene-1,3,5-trisulfonic acid, 7-aminonaphthalene-1,3-disulfonic acid, 7-amino-4-methylcoumarin, alexa fluor hydrazides, BODIPY aliphatic amines, aminomethylfluoresceins, dapoxyl (2-aminoethyl)sulfonamide, dansyl ethylenediamine, dansyl cadaverine, and dapoxyl (2-aminoethyl)sulfonamide, EDANS, lissamine rhodamine B ethylenediamine, fluorescein cadaverine, 1-pyrenemethylamine, Oregon Green 488 (2′,7′-difluorofluorescein), carboxylic acid derivatives of Oregon Green 488, Oregon Green 488 cadaverine, QSY 7 amine, QSY 35 methylamine, Texas Red cadaverine, BODIPY TR cadaverine, tetramethylrhodamine cadaverine, biotin cadaverines, and hydrazine and amine derivatives of Lucifer Yellow, Cascade Blue, biotin, and desthiobiotin.

### Carbodiimide-Mediated Coupling

In yet another example, a carbodiimide-mediated coupling can be used to form a bond between the linking moiety of the adapter antigen and a cell-surface substituent. For example, an adapter antigen with a hydrazine or amine linking moiety can be coupled to cell-surface proteins with carboxylate or carboxylic acid functional groups using water-soluble carbodiimides such as 1-ethyl-3-(3-dimethylaminopropyl)carbodiimide.

Suitable adapter antigens with linking moieties capable of carbodiimide-mediate coupling to carboxylate or carboxylic acid containing cell-surface substituents are commercially available from, for example, Molecular Probes (Eugene, Oreg.). Specific examples of such adapter antigens include, but are not limited to, 2-aminoacridone, 5-aminoeosin, 5-(aminoacetamido)fluorescein, 7-aminonaphthalene-1,3,5-trisulfonic acid, 7-aminonaphthalene-1,3-disulfonic acid, 7-amino-4-methylcoumarin, alexa fluor hydrazides, BODIPY aliphatic amines, aminomethylfluoresceins, dapoxyl (2-aminoethyl)sulfonamide, dansyl ethylenediamine, dansyl cadaverine, and dapoxyl (2-aminoethyl)sulfonamide, EDANS, lissamine rhodamine B ethylenediamine, fluorescein cadaverine, 1-pyrenemethylamine, Oregon Green 488 cadaverine, QSY 7 amine, QSY 35 methylamine, Texas Red cadaverine, BODIPY TR cadaverine, tetramethylrhodamine cadaverine, biotin cadaverines, and hydrazine and amine derivatives of Lucifer Yellow, Cascade Blue, biotin, and desthiobiotin.

In an alternative embodiment involving a carbodiimide-mediated coupling, an adapter antigen with a carboxylate or carboxylic acid lining moiety can be coupled to cell-surface proteins with amine functional groups using water-soluble carbodiimides such as 1-ethyl-3-(3-dimethylaminopropyl)carbodiimide. Suitable adapter antigens with linking moieties capable of carbodiimide-mediate coupling to amine containing cell-surface substituents are commercially available from Molecular Probes (Eugene Oreg.).

### Enzyme-Catalyzed Coupling

In still another example, the adapter antigen can be attached to a cell surface by using a special enzyme-catalyzed transamidation reaction. With transamidation of cell-surface glutamine residues, a transglutaminase enzyme replaces the NH₂ group of glutamine residues in a cell-surface protein or peptide with another amine containing molecule to form a labeled glutamine amide. In this way, an adapter antigen with an amine-containing linking moiety can be bound to a cell surface glutamine residue.

Suitable adapter antigens with linking moieties capable of being coupled to cell-surface glutamine residues with a transglutaminase residue are commercially available from, for example, Molecular Probes (Eugene, Oreg.). Specific examples of such adapter antigens include, but are not limited to, 2-aminoacridone, 5-aminoeosin, 5-(aminoacetamido)fluorescein, 7-aminonaphthalene-1,3,5-trisulfonic acid, 7-aminonaphthalene-1,3-disulfonic acid, 7-amino-4-methylcoumarin, BODIPY aliphatic amines, aminomethylfluoresceins, dapoxyl (2-aminoethyl)sulfonamide, dansyl ethylenediamine, dansyl cadaverine, and dapoxyl (2-aminoethyl)sulfonamide, EDANS, lissamine rhodamine B ethylenediamine, fluorescein cadaverine, 1-pyrenemethylamine, Oregon Green 488 cadaverine, QSY 7 amine, QSY 35 methylamine, tetramethylrhodamine cadaverine, Texas Red cadaverine, BODIPY TR cadaverine, biotin cadaverines, and amine derivatives of Lucifer Yellow, Cascade Blue, biotin, and desthiobiotin.

### Antibodies That Specifically Bind Adapter Antigens

The term “antibodies” is used herein in a broad sense and includes both polyclonal and monoclonal antibodies. In addition to intact immunoglobulin molecules, also included in the term “antibodies” are fragments of immunoglobulin molecules and multimers of immunoglobulin molecules (e.g., diabodies, triabodies, and bi-specific and tri-specific antibodies, as are known in the art; see, e.g., Hudson and Kortt, J. Immunol. Methods 231:177-189, 1999), fusion proteins containing an antibody or antibody fragment, which are produced using standard molecular biology techniques, single chain antibodies, and human or humanized versions of immunoglobulin molecules or fragments thereof. Any antibody that specifically binds an adapter antigen in a manner sufficient to specifically affix a cell labeled with the adapter antigen to a substratum can be used in the methods of the invention.

Whenever possible, the antibodies of the invention may be purchased from commercial sources. The antibodies of the invention may also be generated using well-known methods. The skilled artisan will understand that either full-length adapter antigens or fragments thereof may be used to generate the antibodies of the invention. A polypeptide to be used for generating an antibody of the invention may be partially or fully purified from a natural source, or may be produced using recombinant DNA techniques or solid phase peptide synthesis, using well-known techniques. For example, for adapter antigens that are peptides or polypeptides, a cDNA encoding an adapter antigen, or a fragment thereof, can be expressed in prokaryotic cells (e.g., bacteria) or eukaryotic cells (e.g., yeast, insect, or mammalian cells), after which the recombinant protein can be purified and used to generate a monoclonal or polyclonal antibody preparation that specifically binds the adapter antigen used to generate the antibody.

One of skill in the art will know how to choose an antigenic peptide for the generation of monoclonal or polyclonal antibodies that specifically bind adapter antigens. Antigenic peptides for use in generating the antibodies of the invention are chosen from non-helical regions of the protein that are hydrophilic. The PredictProtein Server (http://www.embl-heidelberg.de/predictprotein/subunit_def.html) or an analogous program may be used to select antigenic peptides to generate the antibodies of the intention. In one example, a peptide of about fifteen amino acids may be chosen and a peptide-antibody package may be obtained from a commercial source such as AnaSpec, Inc. (San Jose, Calif.). One of skill in the art will know that the generation of two or more different sets of monoclonal or polyclonal antibodies maximizes the likelihood of obtaining an antibody with the specificity and affinity required for its intended use (e.g., affixing adapter-antigen-labeled cells to a substratum). The antibodies are tested for their desired activity by known methods (e.g., but not limited to, ELISA and/or immunocytochemistry). For additional guidance regarding the generation and testing of antibodies, see, e.g., Harlow and Lane, Antibodies: A Laboratory Manual, Cold Spring Harbor Laboratory Press, Cold Spring Harbor, N.Y., 1988).

Monoclonal Antibodies

The term “monoclonal antibody” as used herein refers to an antibody or antibody fragment obtained from a substantially homogeneous population of antibodies or antibody fragments, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, e.g., U.S. Pat. No. 4,816,567 and Morrison et al., Proc. Natl. Acad. Sci. USA, 81:6851-6855, 1984).

Monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to, elicit lymphocytes that produce or are capable of producing antibodies that will specifically bind to the immunizing agent. Alternatively, the lymphocytes may be immunized in vitro, e.g., using an adapter antigen or an immunogenic fragment thereof.

The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 (Burton et al.) and U.S. Pat. No. 6,096,441 (Barbas et al.). Recombinant antibodies, antibody fragments, and fusions and polymers thereof can be expressed in vitro or in prokaryotic cells (e.g., bacteria) or eukaryotic cells (e.g., yeast, insect, or mammalian cells) and further purified, as necessary, using well known methods (see, e.g., Sambrook et al. Molecular Cloning: a Laboratory Manual, 3d Edition, Cold Spring Harbor Laboratory Press (2001); and Ausubel et al., Current Protocols in Molecular Biology, John Wiley & Sons, New York, N.Y., 2001, which is updated quarterly).

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

Any antibody or antibody fragment of the invention, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, e.g., to remove or add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified and/or improved by mutagenesis of a specific region of the protein, followed by expression and testing of the expressed polypeptide. For example, amino acid sequence variants of antibodies or antibody fragments can be generated and those that display equivalent or improved affinity for antigen can be identified using standard techniques and/or those described herein. Methods for generating amino acid sequence variants are readily apparent to a skilled practitioner in the art and can include site-specific mutagenesis or random mutagenesis (e.g., by PCR) of the nucleic acid encoding the antibody or antibody fragment (Zoller, M. J. Curr. Opin. Biotechnol. 3:348-354, 1992). Both naturally occurring and non-naturally occurring amino acids (e.g., artificially-derivatized amino acids) may be used to generate amino acid sequence variants of the antibodies and antibody fragments of the invention.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991; and C. F. Barbas, D. R. Burton, J. K. Scott, G. J. Silverman, Phage Display: A Laboratory Manual, Cold Spring Harbor Laboratory Press, Cold Spring Harbor, N.Y., 2001).

The human antibodies of the invention can also be obtained from transgenic animals. For example, transgenic, mutant mice that are capable of producing a full repertoire of human antibodies in response to immunization have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### EXAMPLES

The following examples are put forth so as to provide those of ordinary skill in the art with a complete disclosure and description of how the methods, processes, polypeptides, nucleic acids, and/or compositions claimed herein can be made and evaluated, and are intended to be purely exemplary of the disclosed subject and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, desired solvents, solvent mixtures, temperatures, pressures, and other ranges and conditions that can be used to optimize the methods described herein. Only reasonable and routine experimentation will be required to optimize such process conditions.

### Example I

### Materials and Methods

Reagents. Bodipy-FL-STP and Texas-Red-STP were obtained from Molecular Probes, Eugene, Oreg.; NHS-biotin and neutravidin were obtained from Pierce Chemical Co., Rockville, Ill. Bodipy-FL-STP, Texas-Red-STP, and NHS-biotin were prepared as 5 mg/ml solutions in anhydrous dimethylsulfoxide (DMSO). The fluorophores were protected from light at all times. Just prior to use, the labeling reagents were diluted in Minimal Attachment Medium (MAM; a saline solution that lacks nucleophilic reagents; see Klebe, “Isolation of a collagen-dependent cell attachment factor.” Nature, 250: 248-251, 1974) and used to treat cells immediately. Bovine serum albumin (BSA) (cat. #A7906) and Sepharose-CL-4B-200 were obtained from Sigma Chemical Co., St. Louis, Mo. All other chemicals were of reagent grade.

Labeling cells with “adapter” antigens. Cells were maintained in 50% Dulbecco's Modified Eagle's Medium (MEM)/50% Ham's F-12 containing 10% newborn calf serum plus 100 units/ml penicillin, 100 μg/ml streptomycin, and 50 μg/ml gentamicin sulfate (termed “culture medium” hereafter). MG63 osteosarcoma cells were obtained from the American Type Culture Collection, Manassas, Va.

To attach fluorophores to the cell surface, cells were suspended by trypsinization and washed three times (by centrifugation at 300× g for 3 min) with MAM and treated with 25 μg/ml (in MAM) of the N-hydroxysuccinimide (NHS) or 4-sulfo-2,3,5,6-tetrafluorophenol (STP) active ester labeling reagent for 30 min at 37° C. under conditions that protected the cells from light. After three (or more) washes to remove unbound fluorophore (i.e., adapter antigen), labeled cells were added to substrata coated with antibodies.

It is important that cells be washed free of unreacted fluorophore prior to incubating the cells with the antibody-coated substrate, because the free fluorophore will compete with the labeled cells for binding to the antibody. A long-wavelength ultraviolet light hand lamp can be used to monitor wash solutions to be sure that unreacted fluorophore is completely removed.

Monoclonal antibodies. Hybridomas were prepared according to standard methods (Taggart, K. T. and Samloff, M. “Stable antibody producing murine hybridomas,” Science, 219:1228-1230, 1983), using NS-1 as the parental cell line, and hybridomas were cloned and maintained in the culture medium described above. Monoclonal antibody was affinity-purified on affinity columns of 3,3′-Iminobispropylamine-Sepharose coupled with fluorophore using 0.1 M citric acid, pH 3.0, to elute the antibody. In brief, the affinity support was prepared with Sepharose, which was activated and washed on a sintered glass funnel. Each gram of wet Sepharose was coupled to 6.4 μl of 3,3′-Iminobispropylamine (I-7006, Sigma Chemical Co., St. Louis, Mo.) by cyanogen bromide activation (March et al. “A simplified method for cyanogen bromide activation of agarose for affinity chromatography.” Analyt. Biochem., 60: 149-152, 1974). The volume of 3,3′-Iminobispropylamine employed was chosen to avoid adding an excess number of positively charged moieties to the affinity support which would non-specifically adsorb proteins. The 3,3′-Iminobispropylamine-Sepharose gel was stored in 0.2 M bicarbonate, pH 8.9 at 0.12 ml packed gel/ml of gel suspension. 16 ml of suspended 3,3′-Iminobispropylamine-Sepharose gel was reacted for 1 hr with 6 μl of fluorophore active ester derivative, washed five times with 100 ml of 0.1 M Imidazole, pH 6.95, plus 1 M NaCl and washed and then stored in 0.2 M NaHCO₃ protected from light.

Purification of monoclonal antibody was monitored by enzyme-linked immunosorbent assay (ELISA) according to standard methods, with the antigen being fluorophore-coupled to BSA (3.5 μg/well). A predetermined volume of antibody containing solution was incubated with the affinity reagent for 1 hr, washed with 0.1 M Imidazole, pH 6.95, plus 0.5 M NaCl, and then eluted with 0.1 M citric acid, pH 3.0. The eluted monoclonal antibody was immediately added to 0.1 M Tris, pH 8.5 to adjust the final pH to about pH 8. After ELISA determination, the antibody was concentrated with a Centriplus-30 (Millipore Corp., Billerica, Mass.) to a titer of >10,000.

Micropositioning cells with inkjet printers. In brief, adapter antibodies or other cell-adhesive substances were deposited in desired patterns on a substratum using an inkjet printer. For cells to be micropositioned using the adapter antigen method, cells were derivatized with adapter antigens, after which the derivatized cells were incubated with antibody-coated substrata to allow attachment. Details are presented below.

Both piezoelectric (Epson) and bubblejet (Hewlett-Packard, Canon, etc.) inkjet printers were used to deposit proteins onto substrata. Protein solutions (e.g., adapter antibodies or cell adhesion molecules) were introduced into the printhead in several fashions. Small volumes (˜50 μl) of solution were used by affixing a plastic pipet tip to the inlet port of a printer. Prior to use, the printhead was flushed with water (or dimethylsulfoxide if dried ink was to be removed) to remove all traces of ink. The printhead was primed with 150 μl of the desired solution by printing twenty 600 mm² solid squares at a rate of one square/2 min. The priming procedure ensured that the previous solution has been flushed from the printhead and all nozzles of the printhead were fully primed Protein solutions were prepared in 0.15 M NaCl+0.1 M Imidazole, pH 6.95+0.01% phenol red.

The optimal concentration of antibody or other cell adhesive material for use in any given situation can be empirically determined using routine approaches, as will be understood by one of ordinary skill in the art. For example, antibodies with a titer of >1:1200 and cell adhesion molecules at a concentration of >10 μg/ml were used in the present methods. In some cases, antibodies in ascites fluids required purification by affinity chromatography on antigen-Sepharose to prevent impurities in the ascites fluid from blocking binding of antibody to substrata.

A test run was conducted, e.g., using typewriter paper as a substratum (the phenol red turned yellow on many papers and was made more visible by exposing the paper to NH₄OH vapor). If parts of the image did not appear (indicating clogging of nozzles), the head cleaning utility of the printer was used to open inoperative nozzles. The priming procedure described above generally ensured that all nozzles were fully primed. Following use, the printhead was flushed with distilled water.

Cells were derivatized with adapter antigens as described above and protected from light by covering test tubes with aluminum foil and carrying out all procedures under reduced lighting conditions. Substrata were printed with antibodies or other proteins at appropriate concentrations as described above. The use of polystyrene film (Mobile Chemical Co., Covington, Ga.) and porous sheets of polylactic acid was used in this particular example. However, any plastic that will adsorb proteins (see, e.g., Klebe et al. “Adhesive substrates for fibronectin.” J. Cell. Physiol., 109: 481-488, 1981; Bentley and Klebe, “Fibronectin binding properties of bacteriological Petri plates and tissue culture dishes.” J. Biomed. Mater. Res., 19: 757-769, 1985) can be used in the methods of the invention, as long as the plastic is flexible enough to pass through the rollers of the inkjet printer paper handling apparatus.

The flexible substrata were affixed to plastic Petri plates with transparent tape (e.g., SCOTCH tape, (3M, St. Paul, Minn.)). Following printing with antibodies, the substratum was inactivated to prevent the binding of other proteins by incubation with heat-inactivated BSA in MAM (for heat inactivation, a 10 mg/ml BSA solution was heated to 80° C. in a microwave oven and rapidly cooled on ice). Cells were added at approximately 2×10⁵ cells/5 ml of MAM and agitated at 2 min intervals for 5 sec to attain saturation of the antibody-treated areas with cells. A GraLab451 intervalometer (Cole-Parmer Co., Vernon Hills, Ill.) was used to control a Titertek DSG304 shaking table (Flow Laboratories, McLean Va.) to automate the agitation step. After 10 min, unattached cells were decanted and culture medium was added. Cells became completely spread within approximately 20 min.

### Example II

### Adapter Antigen Approach for Cell Micropositioning

A strategy was developed that allowed simultaneous, specific attachment of multiple cell types to selected sites on a substratum in a single step. The strategy involved derivatizing each type of cell to be micropositioned with a foreign antigen (an “adapter antigen”). A molecule that specifically binds the adapter antigen (e.g., an adapter antibody or other adapter antigen-binding molecule) was deposited onto the substrate in a desired pattern (e.g., using an inkjet printer), after which the cells were allowed to attach to the substrate. Different antibodies that specifically recognize different adapter antigens were simultaneously deposited onto a substratum using an inkjet printer (or analogous means for depositing polypeptides onto a substratum), and the different cell types were simultaneously bound to the substrate, since each type of adapter antigen-derivatized cell bound only to its cognate antibody. For example, cells derivatized with Bodipy-FL or fluorescein by treatment with the active esters of the above fluorophores specifically attached to antibodies to the above fluorophores without the need to inhibit the attachment to either cell-adhesive material (as in the sequential approach; see below).

Simultaneous attachment of selective ligands. In the experiments described below, MG63 cells were derivatized with adapter antigens as described in Example I above. Coupling of fluorophores to cells was easily monitored by examination under a fluorescence microscope. After coupling of adapter antigens, cells were washed extensively to eliminate free ligand which could compete with cell-bound ligand.

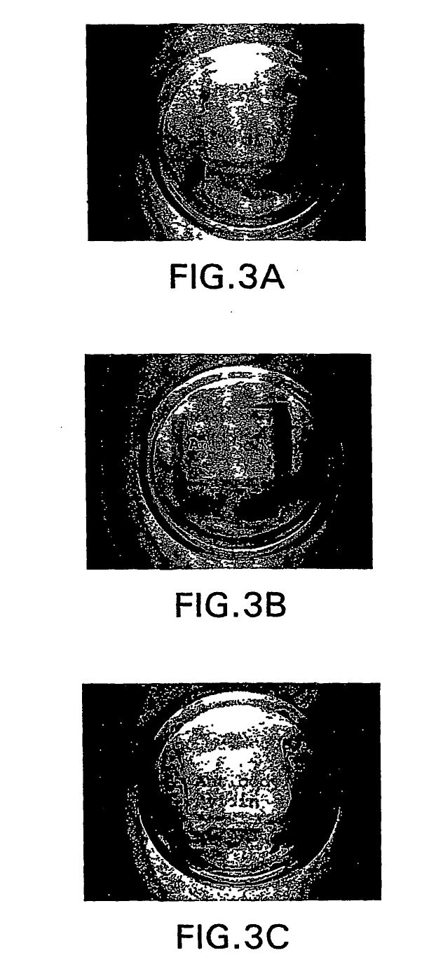

The specificity of binding of cells to the intended cell-adhesive material was assessed by depositing neutravidin or a monoclonal antibody to fluorescein onto substrata and then determining the number of cells that bound to the appropriate antibody. Using an inkjet printer as described above to print on strips of polystyrene film, neutravidin was used at 0.1 mg/ml to print the word “Avidin” (FIG. 3A), and a 1/25 dilution of a monoclonal antibody to fluorescein with a titer of 1:40,000 was used to print the word “Antibody” (FIG. 3B). Cells labeled with the adapter antigens fluorescein or biotin were added simultaneously to a printed substratum that had been inactivated with heat-treated BSA to prevent non-specific binding. Each population of labeled cells bound only to the appropriate adapter antibody or neutravidin (FIG. 3A-3C).

Since one can distinguish between cells labeled with fluorophores by inspection under the fluorescence microscope, the percentage of cells that bound to the appropriate antibody were determined and it was found that the vast majority (>99%) bound to the intended site. Once attached to antibody or neutravidin, cells remained attached to the intended site for at least one day, after which cells started to migrate on the substratum. Cells labeled with fluorescein were prevented from attaching to anti-fluorescein monoclonal antibody by the addition of free fluorescein.

Preparation of three-dimensional (3D) constructs. As described above, addition of adapter antigens to cells permits rapid and highly specific attachment of cells to two-dimensional substrata. The same strategy could be used to prepare three-dimensional constructs of cells. By adding two adapter antigens to a single cell, one can attach the cell to a substratum with the first adapter antigen, and then use the first layer of cells as a platform for constructing a second layer of cells.

Cells used for constructing a first layer were labeled with two adapter antigens, namely, fluorescein and biotin. The first layer was constructed by treating a plastic petri plate with 0.3 ml of 0.1 mg/ml neutravidin and adding 10⁶ double-labeled cells. After 30 min, the first layer had attached and spread. The first layer was then treated with dilutions of an anti-fluorescein monoclonal antibody ranging from a 1/100 to a 1/25 dilution of ascites fluid with a titer of 1:40,000. After the unbound antibody was removed by washing, cells labeled with only a fluorescein adapter antigen were added (these cells were also treated with 1 mg/ml fluorescein diacetate to make them intensely fluorescent). It was found that the number of cells forming the second layer depended on both the amount of adapter antigen used to derivatize the second population of cells and the amount of anti-fluorescein monoclonal antibody applied to the first layer of cells.

### Example III

### Sequential Approach for Cell Micropositioning

For the sequential approach, methods were devised that permit one to add different cell types, one type at a time, to the substratum. The sequential strategy relies on the sequential, selective inhibition of cell adhesion to given cell-adhesive materials. For example, cell adhesion to fibronectin can be inhibited at low temperature (Bentley and Klebe, “Fibronectin binding properties of bacteriological petri plates and tissue culture dishes.” J. Biomed. Mater. Res., 19: 757-769, 1985), whereas cell attachment to plant lectins can be inhibited by simple sugars, and cell attachment to neutravidin only takes place if a cell is derivatized with biotin.

In this particular example of the sequential approach, poly-L-lysine (0.25 mg/ml), fibronectin (0.3 mg/ml), concanavalin-A (Con-A; 0.5 mg/ml), and neutravidin (0.1 mg/ml) were deposited onto substrata as 10 microliter droplets (note that poly-L-lysine must not be diluted in solutions containing phenol red, because these compounds interact and precipitation occurs). Low temperature incubation and mannose were used to inhibit, respectively, fibronectin and Con-A. Since cells do not attach to neutravidin without biotinylation, biotinylated cells were added last and attached only to sites coated with neutravidin. Cells will attach to poly-L-lysine under all conditions and, thus, cells intended to attach to deposited poly-L-lysine were added first. To saturate all regions to which poly-L-lysine was bound (and to reduce the number of cells needed), the substratum was agitate by activation of a Titertek DSG304 shaker table (Flow Laboratories, McLean, Va.) under the control of a Gralab451 intervalometer (Cole-Parmer Co., Vernon Hills, Ill.) which activated the shaker table for 5 sec every 2 min. Cells attached to poly-L-lysine within 2 min and spread within 10 min. Once all poly-L-lysine sites were covered with cells (usually within 10 min), the residual cells were removed and the inhibition of fibronectin was relieved by warming the substratum to 37° C. Again, a regime of repeated agitation was used to ensure attachment of cells to all fibronectin-coated sites. Following attachment of cells to fibronectin, attachment of cells to Con-A was activated by washing the cells three times at room temperature with mannose-free MAM (Minimal Attachment Medium). Lastly, biotinylated cells were added which specifically attach to sites coated with neutravidin (note that it is preferable to use neutravidin, rather than avidin, because avidin can non-specifically bind to cells).

### Incorporation by Reference

Throughout this application, various publications, patents, and/or patent applications are referenced in order to more fully describe the state of the art to which this invention pertains. The disclosures of these publications, patents, and/or patent applications are herein incorporated by reference for the subject matter for which they are specifically referenced in the same or a prior sentence, to the same extent as if each independent publication, patent, and/or patent application was specifically and individually indicated to be incorporated by reference.

### Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for positioning cells onto a substratum, comprising:
a) depositing onto the substratum an adapter antigen-binding molecule; and
b) contacting the substratum with an adapter antigen-labeled cell, thereby positioning the cell onto the substratum.

2. The method of claim 1, wherein the adapter antigen-binding molecule is deposited onto the substratum in a pattern.

3. The method of claim 1, wherein the adapter antigen-binding molecule comprises a polypeptide.

4. The method of claim 3, wherein the adapter antigen-binding molecule comprises an antibody.

5. The method of claim 3, wherein the adapter antigen-binding molecule comprises avidin, neutravidin or streptavidin.

6. The method of claim 1, wherein the adapter antigen comprises a peptide, a polypeptide, biotin, a fluorophore, a hapten, or a carbohydrate.

7. The method of claim 1, wherein the adapter antigen-labeled cell comprises two different adapter antigens.

8. The method of claim 1, wherein at least two different adapter antigen-binding molecules are deposited onto the substratum.

9. The method of claim 1, wherein at least two layers of cells are deposited onto the substratum.

10. The method of claim 1, wherein the adapter antigen-binding molecule is deposited onto the substratum using an inkjet printer.

11. A method of positioning cells onto a substratum, comprising:
a) depositing onto the substratum at least two different cell-adhesive substances;
b) inactivating all but one of the cell-adhesive substances to provide one active cell-adhesive substance and at least one inactive cell-adhesive substance deposited onto the substratum;
c) contacting the one active cell-adhesive substance with a cell, thereby affixing the cell to the substratum;
d) re-activating an inactive cell-adhesion substance; and
e) contacting the re-activated cell-adhesive substance with a cell, thereby affixing the cell to the substratum, thereby positioning cells onto the substratum.

12. The method of claim 11, wherein the cell-adhesive substances are deposited onto the substratum in a pattern.

13. The method of claim 11, further comprising removing unbound cells prior to re-activating an inactive cell-adhesion substance.

14. The method of claim 11, wherein at least three different cell-adhesive substances are deposited onto the substratum, and wherein step d) is repeated to re-activate a third cell-adhesive substance, and wherein step e) is repeated to affix a cell to the substratum via the third cell-reactive substance.

15. The method of claim 14, wherein at least four different cell-adhesive substances are deposited onto the substratum, wherein step d) is repeated to re-activate a fourth cell-adhesive substance, and wherein step e) is repeated to affix a cell to the substratum via the fourth cell-reactive substance.

16. A monoclonal antibody that specifically binds to Bodipy-FL, wherein the monoclonal antibody has the same binding specificity as the monoclonal antibody produced by hybridoma cell line BFL-F12.

17. A hybridoma cell line that produces a monoclonal antibody that specifically binds to Bodipy-FL, wherein the monoclonal antibody has the same binding specificity as the monoclonal antibody produced by hybridoma cell line BFL-F12.

18. A method of binding an antibody to Bodipy-FL, comprising contacting Bodipy-FL with a monoclonal antibody that specifically binds to Bodipy-FL, wherein the monoclonal antibody has the same binding specificity as the monoclonal antibody produced by hybridoma cell line BFL-F12, thereby binding the antibody to Bodipy-FL.
